(19) United States
(12) Patent Application Publication
(10) Pub. No.: US 2009/0076321 A1
(43) Pub. Date: Mar. 19, 2009

(54) DIGITAL CAMERA FOR TAKING IMAGE INSIDE ORAL CAVITY

(75) Inventors: Yuji Suyama, Chiba (JP); Kenji Ito, Chiba (JP)

Correspondence Address: Young & Thompson, 745 S. 23rd Street., Second Floor, Arlington, VA, 22202, US

(73) Assignee: KANAGAWA FURNITURE CO., LTD., Funabashi-shi, Chiba (JP)

(21) Appl. No.: 11/914,613

(22) PCT Filed: Dec. 13, 2006
(86) PCT No.: PCT/JP2006/324842

§ 371 Date: Dec. 11, 2007

(30) Foreign Application Priority Data
Dec. 26, 2005 (JP) 2005-372500

(51) Int. Cl. A61B 1/247

(52) U.S. Cl. 600/109; 600/112; 348/66; 433/29; 348/E05.025

(57) ABSTRACT

To provide a digital camera for taking an image inside the oral cavity without causing a sanitary problem and enabling a dentist to easily monitor an object in the oral cavity of a patient on a screen and the patient to easily view the object on the screen when the camera is used.

A digital camera for taking an image inside the oral cavity comprises a camera body (2) including a lens barrel (2a) to be inserted into the oral cavity, an intermediate part continuous to the lens barrel (2a), and a grip (2b) continuous to the intermediate part; and a dental instrument (3) detachably fitted onto the lens barrel (2a). On the intermediate part of the camera body (2) is mounted a liquid crystal display (2c).

### TECHNICAL FIELD

The present invention relates to a digital camera for photography in the oral cavity, wherein an object in the oral cavity may be displayed on a liquid crystal display device as an still image or moving image by the digital camera which is suitable for dental treatment and dental care.

### BACKGROUND ART

Conventionally, an dentist has inserted a dental unit for oral cavity observation called a dental mirror installing a mirror at the tip of a shaft of about 15 centimeters through 20 centimeters into the oral cavity, and observed an image reflected on the mirror by visual observation for dental treatment and dental care. At this time, the dentist has verbally conveyed the situations to a dental hygienist under waiting and the hygienist has written the situations on a recording form sheet which has been prepared beforehand, wherein a denture mold has been illustrated on the sheet. When the above dental mirror is used for obtaining an informed consent, there has been a problem that it is not easy for the dentist to show a patient the image reflected on the mirror simultaneously while the dentist observes the image.

Moreover, the contents of the results of the dental treatment and those of the dental care are required to accurately and quickly be recorded. But, it is difficult to acquire a dental hygienist recording the contents, or the dental hygienist recording the contents is required to be eliminated for labor savings. Accordingly, various kinds of cameras for photography in the oral cavity have been proposed, and a part of the cameras have been gotten into practical use, wherein an ultrasmall CCD camera is applied to the cameras. But, there has been caused a sanitary problem. The reason is that the camera may not be sterilized by heating under high temperature conditions after using the camera, because the tip portion of the camera is put into the oral cavity of a patient.

Then, the present inventor has proposed a camera unit for medical examination in the oral cavity (Japanese Patent Application Laid-Open No. 2002-143090), wherein, according to the camera, an outer cylinder 13 is installed into a lens barrel portion 12a in a detachable manner as shown in FIG. 10a and FIG. 10b.

The camera unit for medical examination in the oral cavity is provided with: a camera case 12 including the lens barrel portion 12a and a grip portion 12b; the metallic outer cylinder 13 fitting into the lens barrel portion 12a; and a dental unit including heat-resistant glass 13a fitting to the one end of the above outer cylinder 13. According to the configuration in which the above dental unit is provided in a detachable manner, the lens barrel portion 12a to be inserted into the oral cavity may be covered when the camera is used, a part with a greater possibility that the part comes in contact with teeth, the arrangement of the teeth, and the gingivae may be changed for each patient after the camera is used, and the part may be sterilized and disinfected by heating at a heating temperature of, for example, 121 degrees Celsius for heating time of 15 minutes or more, using an autoclave and the like.

Moreover, the camera unit proposed in Japanese Patent Application Laid-Open No. 2002-143090 is provided with a dental mirror attachment 14 shown in FIG. 10a and FIG. 10b, and the dental mirror attachment 14 is preferably made of metal, because the attachment 14 is required to be sterilized and disinfected. The above dental mirror attachment 14 includes: a mirror 14a; a dental mirror holder 14b supporting the mirror 14a; and a mounting and fixing holder 14c mounting and fixing to the dental mirror holder 14b, and the mounting and fixing holder 14c is formed of an elastic and flexible material.

The sanitary problem may be solved by the above-described camera unit for medical examination in the oral cavity.
Patent Document 1: Japanese Patent Application Laid-Open No. 2002-143090

### Disclosure of the Invention

As shown in FIG. 9, a camera case 12 according to Japanese Patent Application Laid-Open No. 2002-143090 includes a lens barrel portion 12a and a grip portion 12b, and the grip portion 12b incorporates: a camera control unit (CCU) 15; and a light source 16 such as a white light emitting diode (LED) for illumination. Furthermore, an optical fiber 17, which may guide illumination light from the light source 16 even to the tip of the lens barrel portion 12a, is disposed on an inner periphery surface in the lens barrel portion 12a, and, at the tip portion of the fiber, there is arranged an objective lens 19, and an image pick up element 18 which opposes to the objective lens 19, and may pick up an image of an object.

Moreover, an image is configured to be displayed on a monitor 21 shown in FIG. 8 when the camera is used, wherein the image represents the condition of an object in the oral cavity of a patient, and is picked tip by the image pick up element 18. In FIG. 8, a cable bundling signal lines and electric power lines supplying electricity to the image pick up element 18, the light source 16, and the like is denoted by 11. An external storage (hard disk) is denoted by 23, and an image printer is denoted by 22.

However, as the camera for medical examination in the oral cavity, described in Japanese Patent Application Laid-Open No. 2002-143090 has a configuration in which the camera is connected to the monitor 21 with the cable 11, and the object in the oral cavity of the patient is displayed on the monitor 21 located at a position different from that of the camera case 12, there has been a problem that a dentist and the patient hardly confirm the condition of the teeth, the arrangement of the teeth, and the gingivae at medical examination.

Considering the above-described conventional technology, an object of the present invention is proposed a digital camera for photography in the oral cavity, wherein the digital camera has no sanitary problem, and, when the camera is used, it is possible for a dentist to easily monitor an object in the oral cavity of a patient, using a screen, and, also, for the patient to easily see the object on the screen.

The present inventors has solved the above-described problem by a configuration in which a similar image pick up system to that of a conventional technology is adopted, and the shape of a camera main body is optimized.

That is, aspects of the present invention are described as follows.

1. A digital camera for photography in the oral cavity, which illuminates an object in the oral cavity, and picks up the image of the object by an image pick up element, including: a camera main body provided with a lens barrel portion to be inserted into the oral cavity, a midway portion formed next to the lens barrel portion, and a grip portion formed next to the midway portion; and a dental unit which is fitted into the lens barrel portion in a detachable manner, wherein a liquid crystal display device is installed in the midway portion of the camera main body.

2. The digital camera for photography in the oral cavity as described in the paragraph 1, wherein the dental unit has at least a metallic outer cylinder, and a heat-resistant glass fitted to the one end side of the outer cylinder, and the outer cylinder may cover the outer periphery surface and the tip surface of the lens barrel portion in a state in which the dental unit is fitted.

3. The digital camera for photography in the oral cavity as described in the paragraph 1 or 2, wherein the grip portion of the camera main body is formed in such a way that the portion is located at one side to the axis center of the lens barrel portion.

4. The digital camera for photography in the oral cavity as described in any one of the paragraphs 1 to 3, wherein a battery, which supplies DC electricity required when the digital camera is used, is incorporated in the grip portion of the camera main body.

5. The digital camera for photography in the oral cavity as described in the paragraph 4, wherein the battery is made a rechargeable type, and there is provided a battery charger having a function by which, when the digital camera is not used, DC electricity is supplied to the rechargeable battery, and, at the same time, the dental unit is preheated.

6. The digital camera for photography in the oral cavity as described in any one of the paragraphs 1 to 5, wherein switches for operation of the digital camera are arranged in the midway portion of the camera main body.

7. The digital camera for photography in the oral cavity as described in any one of the paragraphs 1 to 6, wherein an infrared rays receiving portion is provided in the midway portion of the camera main body, and there is provided an infrared rays remote controller which remotely controls the digital camera through the infrared rays receiving portion.

8. The digital camera for photography in the oral cavity as described in any one of the paragraphs 1 to 7, wherein a recording medium is provided in the camera main body, wherein the medium may be easily detached and attached, and stores image data on an image obtained by picking up with the image pick up element.

9. The digital camera for photography in the oral cavity as described in any one of the paragraphs 1 to 8, wherein a recording microphone is provided in the midway portion of the camera main body, and contents for an informed consent may be digitally recorded.

10. The digital camera for photography in the oral cavity as described in any one of the paragraphs 1 through 9, wherein a lens, in addition to an objective lens, is provided in the lens barrel portion of the camera main body, and the image pick up element of the digital camera is arranged in the midway portion on the extension of the axis center of the lens barrel portion.

According to the digital camera for photography in the oral cavity of the present invention, it is possible for a dentist to easily monitor an object in the oral cavity of a patient, using a screen, and, also, for the patient to easily see the object on the screen when the digital camera is used, because the digital camera has no sanitary problem, and the object in the oral cavity may be displayed on a liquid crystal display device as an still image or moving image.

### BRIEF DESCRIPTION OF DRAWINGS

FIG. 1a is an external view showing a digital camera for photography in the oral cavity according to an embodiment of the present invention in a state in which a dental unit 3 is fitted;

FIG. 1b is an external view showing the digital camera for photography in the oral cavity according to the embodiment of the present invention in a state in which the dental unit 3 is removed;

FIG. 2 is an external view showing a battery charger preferably provided in the digital camera for photography in the oral cavity;

FIG. 3 is an external view showing a an infrared rays remote controller preferably provided in the digital camera for photography in the oral cavity;

FIG. 4 is an explanatory view of a substrate preferably provided in the digital camera for photography in the oral cavity;

FIG. 5 is an explanatory view of an application method of a recording medium preferably provided in the digital camera for photography in the oral cavity;

FIG. 6a is an explanatory view of a configuration example of an image pick up system in the digital camera for photography in the oral cavity;

FIG. 6b is an explanatory view of an image obtained by being picked up, using the configuration example of the image pick up system in the digital camera for photography in the oral cavity;

FIG. 7a is an explanatory view of a configuration example of an image pick up system in the digital camera for photography in the oral cavity;

FIG. 7b is an explanatory view of an image obtained by being picked up, using the configuration example of the image pick up system in the digital camera for photography in the oral cavity;

FIG. 8 is an external view showing a configuration of a conventional camera unit for medical examination in the oral cavity;

FIG. 9 is a cross sectional view showing a configuration for image picking up in the conventional camera unit for medical examination in the oral cavity;

FIG. 10a is an explanatory external view of a configuration of a conventional camera unit for medical examination in the oral cavity; and

FIG. 10b is an explanatory exploded view of the configuration of the conventional camera unit for medical examination in the oral cavity.

### DESCRIPTION OF REFERENCE NUMERALS

1 Axis center of lens barrel portion
2 Camera main body
2a Lens barrel portion
2b Grip portion
2c Liquid crystal display device
2d Supporting portion
2e Battery
2f Infrared rays receiving portion
2g Recording microphone
3 Dental unit
3a Mirror
3b Dental mirror holder
3c Outer cylinder
3d Heat-resistant glass
4 Recording medium (memory card and the like)
5 Battery charger
5a AC adapter
5b Receptacle
6 Infrared rays remote controller
6a Infrared rays emitting portion
7 Image pick up element of C-MOS camera
8 Substrate
8a Insertion portion of recording medium
9 Personal computer
10 Seal printer
10a Seal
11 Cable
12 Camera case
12a Lens barrel portion
12b Grip portion
12c Axis center of lens barrel
13 Outer cylinder
13a Heat-resistant glass
14 Dental mirror attachment
15 CCU
16 White led (light source)
17 Optical fiber
18 Image pick up element of CCD camera
19 Objective lens
20 Protection lens
21 Monitor
22 Image printer (video printer)
23 Storage device (hard disk)
24 Lens

### BEST MODE OF CARRYING OUT THE INVENTION

A configuration of a digital camera for photography in the oral cavity according to an embodiment of the present invention will be explained in detail, referring to drawings.

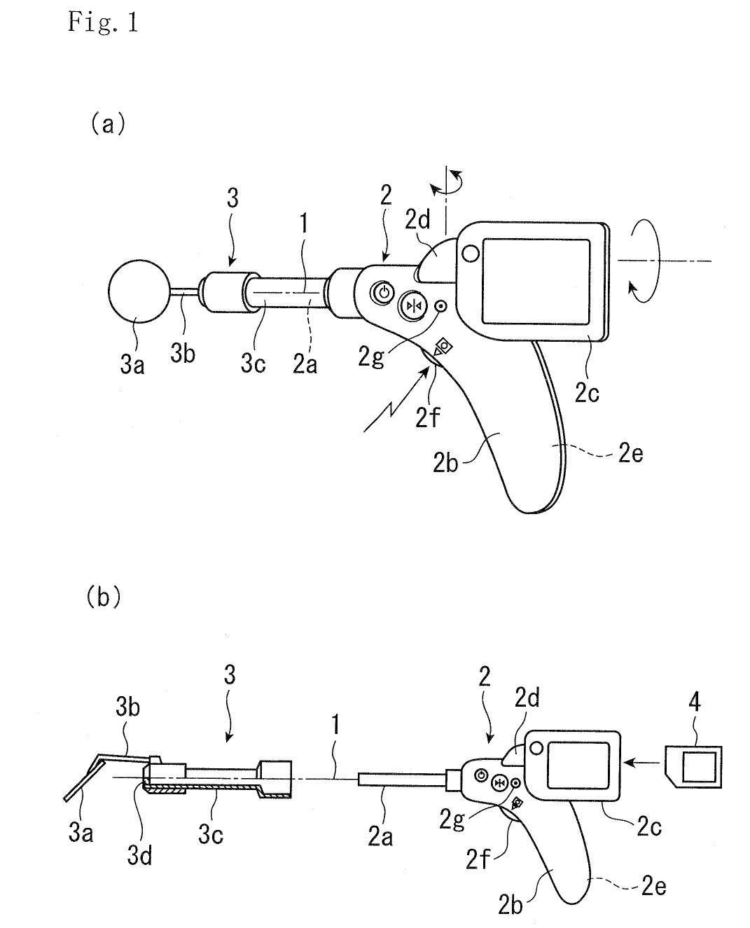

FIG. 1a and FIG. 1b are views of the appearance of the digital camera for photography in the oral cavity for comparison between a state in which a dental unit 3 is fitted into the digital camera for photography in the oral cavity as shown in FIG. 1a, and a state in which the dental unit is removed therefrom as shown in FIG. 1b, and FIG. 1b also includes a partial sectional view.

The digital camera for photography in the oral cavity according to the embodiment of the present invention is provided with a camera main body 2 including a lens barrel portion 2a to be inserted into the oral cavity, and the like, and the dental unit 3 as a dental unit which is fitted into the lens barrel portion 2a of the camera main body 2 in a detachable manner, and observes the other side of the arrangement of the teeth, and a liquid crystal display device 2c is installed in the midway portion of the camera main body 2.

As shown in FIG. 1a and FIG. 1b for comparison, the above camera main body 2 includes; the lens barrel portion 2a to be inserted into the oral cavity, the midway portion formed next to the portion 2a; and a grip portion 2b formed next to the above midway portion, and the digital camera has a configuration in which, when the camera is used, the lens barrel portion 2a may be covered in a state in which the dental unit 3 is fitted. The above dental unit 3 has: a mirror 3a; a dental mirror holder 3b supporting the mirror 3a; and a metallic outer cylinder 3c fitting to the dental mirror holder 3b, and the metallic outer cylinder 3c is an attaching portion to the camera main body 2.

Thereby, the dental unit 3 having the mirror 3a with high possibility that the mirror 3a comes in direct contact with teeth, the arrangement of the teeth, and the gingivae in the oral cavity when the camera is used may be removed from the lens barrel portion 2a of the camera main body 2 after use of the camera, and may be sterilized and disinfected by heating, using an autoclave and the like. Accordingly, the digital camera for photography in the oral cavity according to the embodiment of the present invention may solve the sanitary problem because the dental unit 3 may be changed to another dental unit 3, which has been sterilized and disinfected, for each patient.

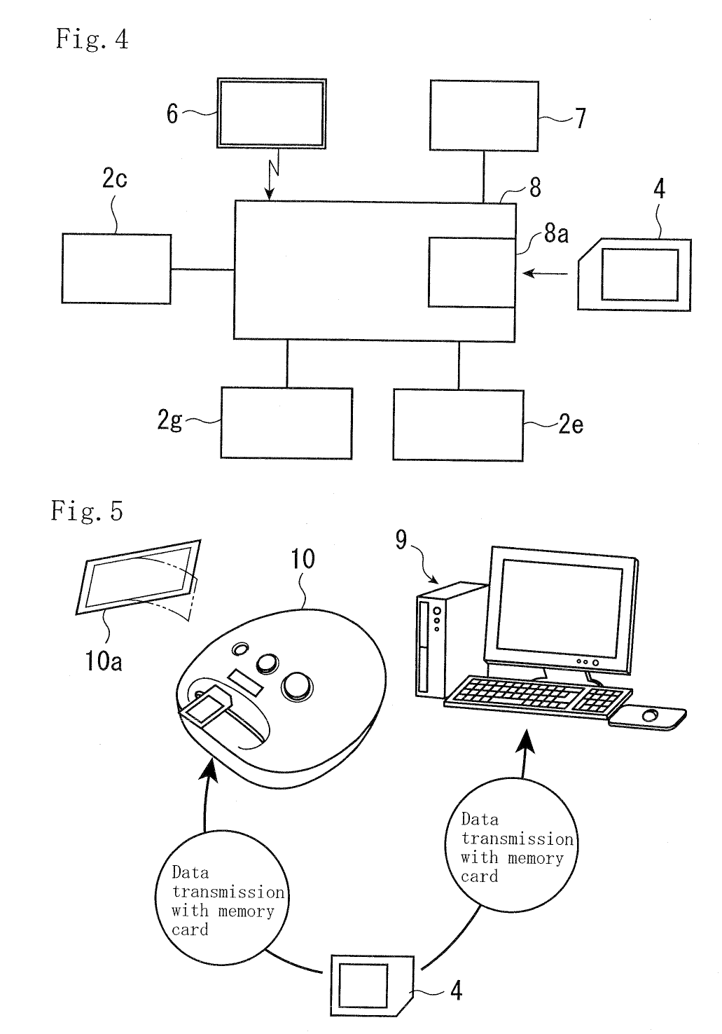

Here, as one of configurations in which an object in the oral cavity is illuminated, and the image of the object is picked up by an image pick up element, there may be adopted a configuration in which for example, a camera control unit (CCU) and a light source such as a white light-emitting diode (LED) for illumination are provided in the camera main body 2, an optical fiber, which guides illumination light from the light source to the tip of the lens barrel portion 2a and an image pick up element are disposed in the lens barrel portion 2a, and an objective lens 19 is fitted into the tip portion of the lens barrel portion 2a. The above-described liquid crystal display device 2C is also connected to CCU through a substrate 8 (refer to FIG. 4). Here, CCU is disposed on the substrate 8 beforehand.

The digital camera for photography in the oral cavity having the above-described image pick up system has a configuration in which, when the camera is used, the illumination light may irradiate the object in the oral cavity of an patient through the dental unit 3, the image of the condition of the teeth, the arrangement of the teeth, and the gingivae of the patient may be picked up by an image pick up element 7 of a complementary metal oxide semiconductor (C-MOS) camera, and the image may be displayed on the liquid crystal display device 2C, which is installed in the midway portion of the camera main body 2, as an still image or moving image.

Here, the liquid crystal display device 2C is installed in the midway portion of the camera main body 2 through a supporting portion 2d as shown in FIG. 1a and FIG. 1b in such a way that the device 2c may be rotated around an axis parallel to an axis center 1 of the lens barrel portion 2a, and around an axis orthogonal to the axis center 1 of the lens barrel portion 2a. Accordingly, the digital camera for photography in the oral cavity according to the embodiment of the present invention has a configuration in which, when the camera is used, the direction of the liquid crystal display device 2C installed in the midway portion of the camera main body may be adjusted, and it is possible for a dentist to easily monitor the object in the oral cavity of the patient, using a screen, and, also, for the patient to easily see the object on the screen.

There is preferably applied a configuration in which the above-described dental unit 3 has at least: the metallic outer cylinder 3c; and a heat-resistant glass 3d fitted to the one end side of the outer cylinder 3c, and the outer cylinder 3c may cover the outer periphery surface and the tip surface of the lens barrel portion 2a of the camera main body 2, as shown in FIG. 1a, in a state in which the dental unit 3 is fitted. According to the digital camera for photography in the oral cavity, which is provided with the above-described dental unit 3, the saliva of the patient may be surely prevented from coming in contact with the lens barrel portion 2a even when the camera comes in contact with teeth, the arrangement of the teeth, and the gingivae in the oral cavity of the patient at use of the camera.

The above-described liquid crystal display device 2c preferably has a screen size of two inches through three inches from the viewpoint of easiness in seeing and handling. Moreover, an image pick up element 7 of a complementary metal oxide semiconductor (C-MOS) color camera of 300,000 pixels or more may be used, considering the resolution and the like. However, the image pick up element used for the digital camera for photography in the oral cavity is not limited to the image pick up element 7 of the C-MOS color camera, but it is acceptable to equip an image pick up element of a charge coupled device (CCD) camera.

Here, FIG. 4 is a schematic view showing a configuration of the substrate 8 provided in the camera main body 2. The image pick up element 7 of the C-MOS color camera, the liquid crystal display device 2c, switches for operation of the digital camera, an infrared rays receiving portion, a battery 2e, a recording microphone 2g, and the like are connected to CCU through the substrate 8. An infrared rays remote controller is denoted by 6, and a signal from the controller is configured to be sent to CCU through the substrate 8. An insertion portion of a recording medium 4 such as a memory card is denoted by 8a, wherein the portion is provided on the substrate 8.

Incidentally, the diameter of the lens barrel portion 2a in the camera main body 2 usually is about 7 mm. Thereby, the midway portion formed between the lens barrel portion 2a and the grip portion 2b has a size and a structure in such a way that there may be installed switches for operation of the liquid crystal display device 2c and of the digital camera operation, the infrared rays receiving portion 2f, the recording microphone 2g, and the like.

Moreover, the digital camera for photography in the oral cavity preferably has a configuration, in which the grip portion 2b of the camera main body 2 is formed next to the midway portion in such a way that the portion 2b is located at one side to the axis center 1 of the lens barrel portion 2a, because the dentist easily holds the camera when the camera is used. Furthermore, the digital camera for photography in the oral cavity preferably has a configuration, in which the front surface of the grip portion is tilted to the side of the dentist in a state in which the grip portion 2b is held in such a way that the lens barrel portion 2a is in a horizontal position, because the dentist easily handles the camera when the camera is used.

Moreover, the digital camera for photography in the oral cavity preferably has a configuration in which the battery 2e, which supplies DC electricity required when the camera is used, is incorporated in the grip portion 2b of the camera main body 2 because the camera may not be required to be connected to the power supply cable, and may be a cordless type, and the dentist easily handles the camera. In the above case, the digital camera for photography in the oral cavity further preferably has a configuration in which the battery 2e is made a rechargeable type, and there is provided a battery charger 5 having a function by which, when the camera is not used, the DC electricity is supplied to the rechargeable battery 2e, and, at the same time, the dental unit 3 is preheated.

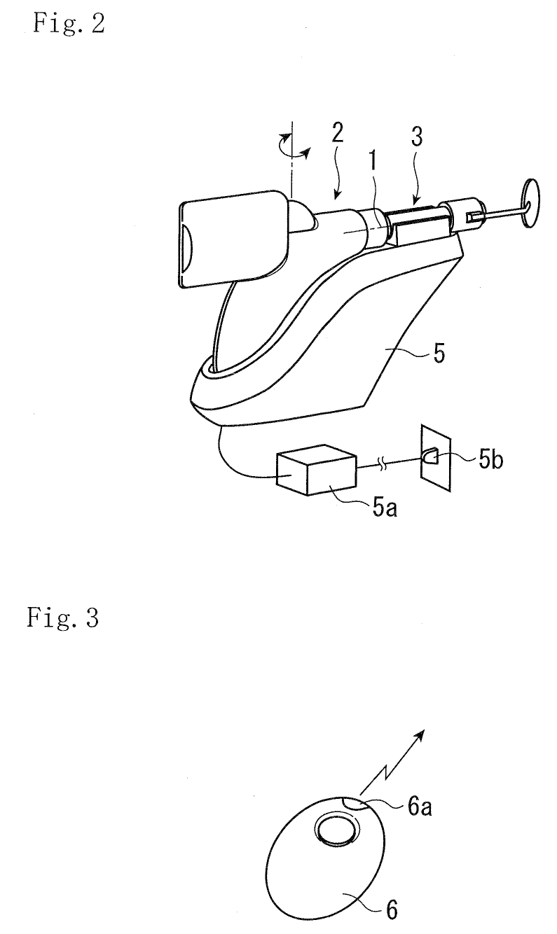

The reason is that the mirror 3a may be prevented from getting clouded when the mirror 3a is inserted into the oral cavity of the patient, based on a configuration in which, when the camera is not used, the camera main body 2 fitting the dental unit 3 may be mounted on the battery charger 5 as shown in FIG. 2 to charge the battery 2e, using the battery charger 5, and, at the same time, CCU incorporated in the camera main body 2 may be operated to conduct the heat of electrical components to the mirror 3a through the metallic outer cylinder 3c for warming the mirror 3a, wherein the heat is caused by the DC electricity consumed by the components.

Here, the digital camera for photography in the oral cavity also preferably has a configuration in which a metallic receiving portion incorporating a heater is provided in the battery charger 5, wherein heat is generated by the DC electricity in the heater, and, when the camera is not used, CCU is operated by bringing the metallic outer cylinder 3c into contact with the metallic receiving portion, and the heat of the heater consuming the DC electricity is conducted to the mirror 3a through the metallic outer cylinder 3c. The reason is that, after the dental unit 3 is warmed by the battery charger 5 when camera is not used, a sense of incompatibility of the patient may be controlled when the dental unit 3 is brought into contact with the oral cavity portion of the patient at use of the camera, wherein the battery charger 5 has the function to preheat the above dental unit 3.

Considering the above situations, the dental unit 3 provided in the digital camera for photography in the oral cavity preferably uses aluminum or aluminum alloy with excellent heat conductivity, as metal In FIG. 2, an AC adapter of the battery charger 5 is denoted by 5a, and a receptacle of the battery charger 5 is denoted by 5b.

Moreover, the digital camera for photography in the oral cavity preferably has a configuration in which the switches for operation of the digital camera are arranged in the midway portion of the camera main body 2, because the dentist easily handles the digital camera. Preferably, the digital camera for photography in the oral cavity preferably has a configuration in which the infrared rays receiving portion 2f is provided in the midway portion of the camera main body 2 as shown in FIG. 1a and FIG. 1b, and there is provided a infrared rays remote controller 6 which remotely controls the digital camera through the infrared rays receiving portion 2f.

The reason is that, when the camera is used, focusing of the digital camera, and starting and ending of image picking up may be controlled by a configuration in which the dental hygienist, instead of the dentist, operates the infrared rays remote controller 6 illustrated in FIG. 3. In FIG. 3, 6a denotes an infrared rays emitting portion which is directed toward the infrared rays receiving portion 2f when the digital camera is remotely operated.

Moreover, the digital camera for photography in the oral cavity preferably has a configuration in which the recording medium 4 is provided in the camera main body 2, wherein the medium 4 may be easily detached and attached, and stores image data on the image obtained by picking up, using the image pick up element. For example, a memory card is used as the recording medium 4. The reason is that, as shown in FIG. 5, image data of the object in the oral cavity of the patient, is stored in a personal computer 9 through the recording medium 4 such as the memory card, wherein the image of the object has been picked up by the digital camera for photography in the oral cavity, and the results of processes image data are output as a seal 10a from a seal printer 10 for application.

The digital camera for photography in the oral cavity preferably has a configuration in which the recording microphone 2g is provided in the midway portion of the camera main body 2, and contents for an informed consent may be digitally recorded, because the dentist may confirm the contents later.

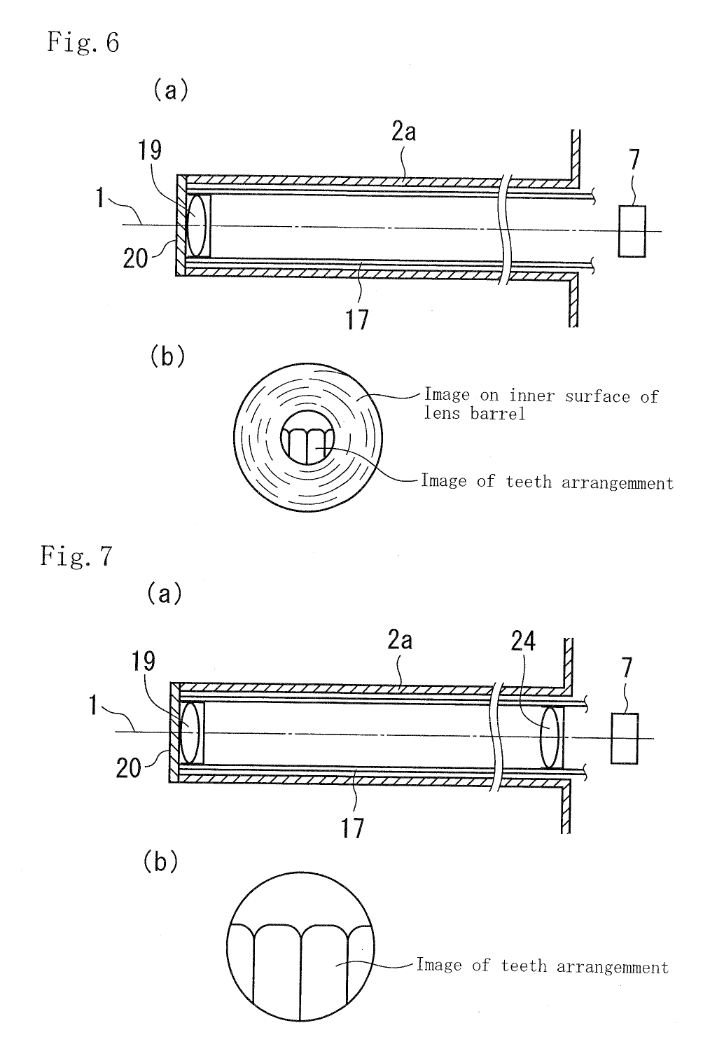

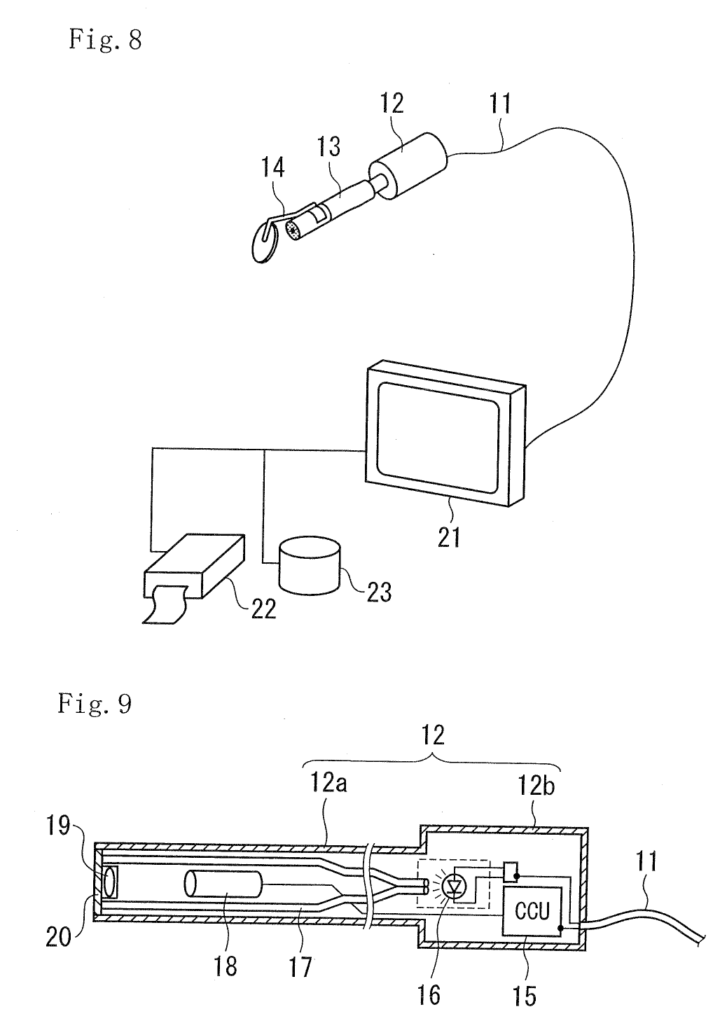

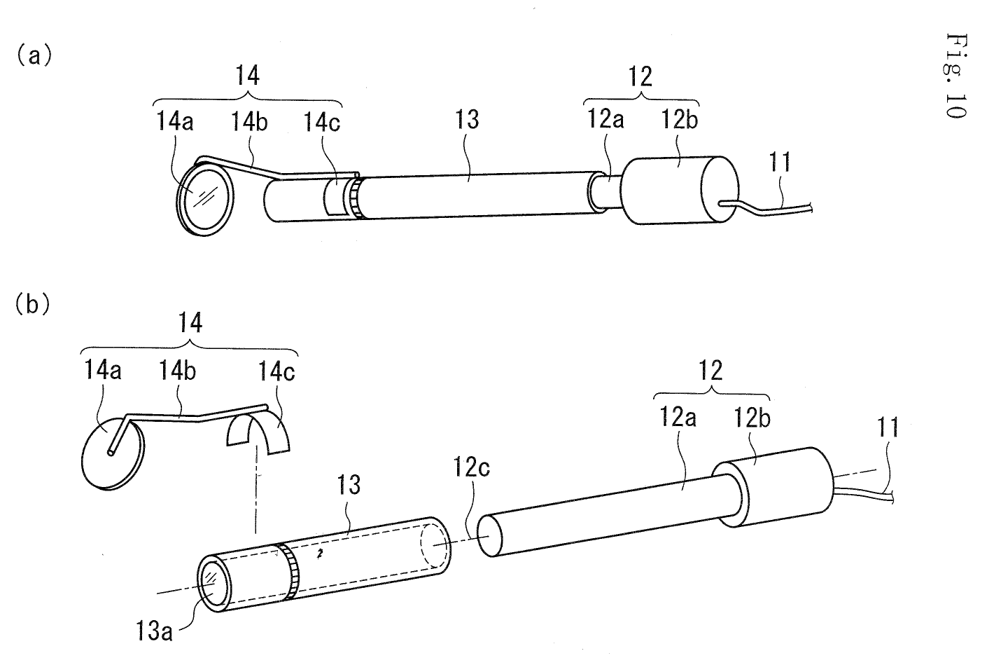

Moreover, the digital camera for photography in the oral cavity preferably has a configuration in which a lens 24, in addition to the objective lens 19, is provided in the lens barrel portion 2a of the camera main body 2 as shown in FIG. 7a, and the image pick up element 7 of the C-MOS camera is arranged in the midway portion on the extension of the axis center 1 of the lens barrel portion 2a. The reason is that, according to the above configuration, an image with a better resolution may be obtained in comparison with the configuration (a case in which the lens 24 is not provided in the lens barrel portion 2a) shown in FIG. 6a. Here, FIG. 6b is a schematic view showing a case in which, based on the configuration shown in FIG. 6a, image data obtained with the image pick up element 7 of the C-MOS camera is not digitally expanded and is displayed on the above-described liquid crystal display device 2C, and FIG. 7b is a schematic view showing a case in which, based on the configuration shown in FIG. 7a, image data obtained with the image pick up element 7 of the C-MOS camera is not digitally expanded and is displayed on the above-described liquid crystal display device 2C. Here, a configuration, in which the image pick up element 7 of the C-MOS camera is disposed in the midway portion on the extension of the axis center 1 of the lens barrel portion 2a, also causes a possibility that the diameter of the lens barrel portion 2a is made thinner in comparison with a convention one.

Though the dental unit 3 has had the mirror 3a in the above-described explanation, the digital camera for photography in the oral cavity according to the present invention may be provided with the dental unit which does not have the mirror 3a. In the above case, it is important to use a dental unit which may be fitted into the lens barrel portion 2a of the camera main body 2 in a detachable manner, has a shape by which the unit may cover the lens barrel portion 2a when the unit is fitted, and is of a material which is less deformed even when the unit is changed for each patient after the unit is used, and the unit is repeatedly sterilized and disinfected by heating, using an autoclave and the like.

1. A digital camera for photography in the oral cavity, which illuminates an object in the oral cavity, and picks up the image of said object by an image pick up element, including:
a camera main body (2) provided with a lens barrel portion (2a) to be inserted into the oral cavity, a midway portion formed next to said lens barrel portion (2a), and a grip portion (2b) formed next to said midway portion; and
a dental unit (3) which is fitted into said lens barrel portion (2a) in a detachable manner, wherein
a liquid crystal display device (2c) is installed in said midway portion of said camera main body (2).

2. The digital camera for photography in the oral cavity according to claim 1, wherein
said dental unit (3) has at least a metallic outer cylinder (3c), and a heat-resistant glass (3d) fitted to the one end side of said outer cylinder (3c), and said outer cylinder (3c) may cover the outer periphery surface and the tip surface of said lens barrel portion (2a) in a state in which said dental unit (3) is fitted.

3. The digital camera for photography in the oral cavity according to claim 1, wherein
said grip portion (2b) of said camera main body (2) is formed in such a way that said portion (2b) is located at one side to the axis center (1) of said lens barrel portion (2a).

4. The digital camera for photography in the oral cavity according to claim 1, wherein,
a battery (2e), which supplies DC electricity required when said digital camera is used, is incorporated in said grip portion (2b) of said camera main body (2).

5. The digital camera for photography in the oral cavity according to claim 4, wherein,
said battery (2e) is made a rechargeable type, and there is provided a battery charger (5) having a function by which, when said digital camera is not used, DC electricity is supplied to said rechargeable battery (2e), and, at the same time, said dental unit (3) is preheated.

6. The digital camera for photography in the oral cavity according to claim 1, wherein,
switches for operation of said digital camera are arranged in said midway portion of said camera main body (2).

7. The digital camera for photography in the oral cavity according to claim 1, wherein,
an infrared rays receiving portion (20) is provided in said midway portion of said camera main body (2), and there is provided an infrared rays remote controller (6) which remotely controls said digital camera through said infrared rays receiving portion (20).

8. The digital camera for photography in the oral cavity according to claim 1, wherein,
a recording medium (4) is provided in said camera main body (2), wherein said medium (4) may be easily detached and attached, and stores image data on an image obtained by picking up with said image pick up element.

9. The digital camera for photography in the oral cavity according to claim 1, wherein,
a recording microphone (2g) is provided in said midway portion of said camera main body 2, and contents for an informed consent may be digitally recorded.

10. The digital camera for photography in the oral cavity according to claim 1, wherein,
a lens (24), in addition to an objective lens (19), is provided in said lens barrel portion (2a) of said camera main body (2), and said image pick up element of said digital camera is arranged in said midway portion on the extension of said axis center (1) of said lens barrel portion (2a).

11. The digital camera for photography in the oral cavity according to claim 2, wherein
said grip portion (2b) of said camera main body (2) is formed in such a way that said portion (2b) is located at one side to the axis center (1) of said lens barrel portion (2a).

12. The digital camera for photography in the oral cavity according to claim 2, wherein,
a battery (2e), which supplies DC electricity required when said digital camera is used, is incorporated in said grip portion (2b) of said camera main body (2).

13. The digital camera for photography in the oral cavity according to claim 2, wherein,
switches for operation of said digital camera are arranged in said midway portion of said camera main body (2).

14. The digital camera for photography in the oral cavity according to claim 2, wherein,
an infrared rays receiving portion (20) is provided in said midway portion of said camera main body (2), and there is provided an infrared rays remote controller (6) which remotely controls said digital camera through said infrared rays receiving portion (20).

15. The digital camera for photography in the oral cavity according to claim 2, wherein,
a recording medium (4) is provided in said camera main body (2), wherein said medium (4) may be easily detached and attached, and stores image data on an image obtained by picking up with said image pick up element.

16. The digital camera for photography in the oral cavity according to claim 2, wherein,
a recording microphone (2g) is provided in said midway portion of said camera main body 2, and contents for an informed consent may be digitally recorded.

17. The digital camera for photography in the oral cavity according to claim 2, wherein,
a lens (24), in addition to an objective lens (19), is provided in said lens barrel portion (2a) of said camera main body (2), and said image pick up element of said digital camera is arranged in said midway portion on the extension of said axis center (1) of said lens barrel portion (2a).
